(19) United States
(12) Patent Application Publication
(10) Pub. No.: US 2026/0298910 A1
(43) Pub. Date: Oct. 1, 2026

(54) IMMUNOGENICITY SCREENING METHOD

(71) Applicant: The Research Foundation for The State University of New York, Amherst, NY (US)

(72) Inventors: Sathy BALU-IYER, Amherst, NY (US); Nicole JARVI, Manlius, NY (US)

(21) Appl. No.: 19/478,694

(22) PCT Filed: Apr. 24, 2024
(86) PCT No.: PCT/US2024/025944

§ 371 Date: Oct. 24, 2025

Related U.S. Application Data
(60) Provisional application No. 63/498,000, filed on Apr. 24, 2023.

(51) Int. Cl. G01N 33/50; G01N 15/14

(52) U.S. Cl. CPC G01N 33/5029; G01N 15/1456; G01N 33/5047; G01N 2015/1486; G01N 2333/521

(57) ABSTRACT

Provided is a method for determining if a protein will produce an immunogenic response. The immunogenic response may be correlated by the migration of dendritic cells in the presence of the protein. The method may be used as a screening tool to assess immunogenicity risk for proteins that could be administered subcutaneously.

### CROSS REFERENCE TO RELATED APPLICATION

This application claims the benefit of U.S. Provisional Patent Application No. 63/498,000, filed on Apr. 24, 2023, the disclosure of which is incorporated herein in its entirety by reference.

### BACKGROUND OF THE DISCLOSURE

Subcutaneous (SC) administration of therapeutic proteins is desirable due to improved cost, convenience, and compliance compared to intravenous (IV) injection; however, there remains a risk of immunogenicity by this route of administration. The SC route introduces unique immunogenicity challenges compared to intravenous delivery, and some therapeutic proteins and monoclonal antibodies (mAb) demonstrate enhanced immunogenicity by SC administration when directly compared to IV. Anti-drug antibodies (ADA) formed against therapeutic proteins, regardless of binding or neutralizing activity, can have a major impact on product safety and efficacy. Immunogenicity can even cause termination of a development program. Thus, to diminish costs of drug development and attrition, companies should attempt to predict immunogenicity risk of therapeutic protein candidates during preclinical development stages. However, in vitro methods for immunogenicity risk assessment remain unstandardized and lack predictive power. ADA development against therapeutic proteins is the consequence of an adaptive immune response driven by antigen-specific interactions between immune cells, namely dendritic cells, T cells, and B cells. Involvement of the T-cell dependent immune response is the rationale for T-cell based immunogenicity risk assessment approaches, including T-cell proliferation/cytokine production assays, in silico screening of T-cell epitopes (T helper and/or Tregitopes), T-cell epitope presentation in MAPPS, and so on. Current gaps in the ability of immunogenicity risk assessment to predict clinical immunogenicity outcomes reveal the insufficiency of available methods. T cell activation and differentiation during the adaptive immune response requires strong, prolonged signals; and with a T-cell focused approach, the early steps in the innate immune response are overlooked. And the early innate immune cell responses, not captured adequately by current risk assessment methods, are particularly important for driving SC immunogenicity of therapeutic proteins.

### SUMMARY OF THE PRESENT DISCLOSURE

The present disclosure provides a method for determining if a protein will produce an immunogenic response. The immunogenic response may be correlated by the migration of dendritic cells in the presence of the protein. The method may be used as a screening tool to assess immunogenicity risk for proteins that could be administered subcutaneously.

A method of the present disclosure comprises determining migration of dendritic cells. A method may comprise contacting a known amount of dendritic cells in the first chamber of the Boyden chamber with a therapeutic protein. The second chamber contains the therapeutic protein and one or more chemokines and does not contain any dendritic cells. Each chamber further comprises serum-free media. The concentration of the therapeutic protein in the first chamber and second chamber is different. The concentration of the therapeutic protein is lower in the first chamber than in the second chamber, such that a concentration gradient is formed between the two chambers. The chambers are incubated for a period of time. During incubation, some of the dendritic cells may migrate from the first chamber into the second chamber. The migrated dendritic cells may then be isolated, stained, and counted. Following counting, the amount of migrated dendritic cells may be used to determine the protein's immunogenicity response/risk. The immunogenicity response/risk may be determined by comparing the percent of dendritic cells migrated to a control.

The migration of may be stimulated by various therapeutic proteins. For example, the therapeutic proteins may be monoclonal antibodies. Additional examples of therapeutic proteins include, but are not limited to, recombinant enzymes, cytokines, globular proteins, AAV capsid proteins, and the like, and combinations thereof.

In an aspect, the present disclosure provides a kit. The kit may provide instructions for use of the kit. The kit may further comprise media, one or more chemokines (e.g., CCL21 and CXCL12), and one or more plates comprising one or more Boyden chambers.

### BRIEF DESCRIPTION OF THE FIGURES

For a fuller understanding of the nature and objects of the disclosure, reference should be made to the following detailed description taken in conjunction with the accompanying figures.

FIG. 1. Flow cytometry gating strategy for human monocyte-derived dendritic cells. Cells were gated based on FSC-A vs SSC-A then singlets were gated based on FSC-A vs FSC-H. Live cells were gated based on low expression of viability dye and then moDC were gated based on expression of CD11c and HLA-DR. Gates for the populations of interest, CXCR4⁺, IL-12⁺, and CD40^{high}, were set based on fluorescence-minus-one control samples.

FIG. 2. CXCR4 is upregulated on dendritic cells concurrently with activation markers CD40 and IL-12 by therapeutic proteins in proportion to their immunogenic potential. Stimulation index values for (a) CXCR4⁻, (b) IL-12⁺ and (c) CD40^{high} (%) of CD11c⁺ moDC populations, respectively, in six healthy donors for anti-TNF IgG1, ATR-107, HuA33, and KLH (mean±SEM). Each dot represents one donor's response (see plot legend), and treatments were tested in triplicate for each donor. Statistical significance was determined by unpaired student's t-test in comparison with the Anti-TNF IgG1 group. *p<0.05, **p<0.01, ***p<0.001.

FIG. 3. The combined readout from the screening assay correlates with immunogenicity incidence for a panel of therapeutic proteins. (a-b) Pooled stimulation index values for all proteins tested during validation where the mean represents the Total Response Index. Errors bars are mean±SEM. Dashed lines are placed at stimulation index equal to 1.0 (no response threshold) and stimulation index equal to 1.7 (positive response threshold set at 80^{th} percentile of anti-TNF IgG1 responses). Statistical significance was determined by unpaired student's t-test. *p<0.05, **p<0.01, ***p<0.001. (c) The Total Response Index for each therapeutic protein, labeled A-H, versus the highest reported clinical ADA incidence in literature or package inserts. The best-fit line based on linear regression is shown. Dashed lines are placed at margins predicting low (green dashed line), mid (orange dashed line), and high immunogenicity risk (red dashed line). A—KLH, B—ATR-107, C—HuA33, D—adalimumab, E—trastuzumab, F—rituximab, G—emicizumab, and H—tocilizumab.

FIG. 4. Immunogenic therapeutic proteins not only upregulate CXCR4 but directly stimulate migration of moDC toward chemokine ligands. (a) Schematic of the Transwell migration assay capturing the potential for dendritic cell migration toward therapeutic protein in the SC space. A concentration gradient of therapeutic protein and chemokines is created across the Transwell insert, immature DCs plated in the upper chamber migrate into the lower chamber, and migrated DCs are counted in the lower chamber by flow cytometry. (b) (Left y-axis) The stimulation index for CXCR4⁺ moDC as a function of KLH concentration. (Right y-axis) The migration index of moDC as a function of KLH concentration in the lower chamber. (c-d) The migration index of moDC along a concentration gradient of therapeutic protein and chemokines in multiple donors. The concentration gradient of therapeutic protein was either (c) 1:10 or (d) 1:5 (at a concentration 5-times lower than that in (c)). The lower chambers contained the same concentration of chemokines in all tests. Each dot represents one donor's response, and treatments were tested in triplicate for each donor. All error bars are mean±SEM. Statistical significance was determined by ordinary one-way ANOVA with Tukey's multiple comparisons test. *p<0.05, **p<0.01, ***p<0.001, ****p<0.0001.

FIG. 5. Following subcutaneous injection of therapeutic protein, the immunogenic response is driven by skin-derived dendritic cell migration into the injection site and toward the draining lymph node. At which point migratory dendritic cells will induce strong cognate T-cell activation.

FIG. 6. CXCR4 induction on human moDC by therapeutic protein correlates with its immunogenicity potential. (A) Flow cytometry gating strategy for live CD11c⁺ moDC. (B) CXCR4 and (C) CD40 stimulation index values in six healthy donors for eight protein antigens (mean±SEM). Each dot represents one donor; treatments were tested in triplicate for each donor. (D) Migration index toward a low protein concentration gradient for six protein antigens in multiple donors (mean±SEM). Migration index equals the percent migrated DC for each treatment group divided by the average percent migrated DC for the unstimulated group. Treatments were tested in triplicate for each donor. Ordinary one-way ANOVA with Tukey's multiple comparisons test. (E) Migration index toward a high protein concentration gradient for three protein antigens in two donors. Unpaired student's t-test. *p<0.05, **p<0.01, ***p<0.001.

FIG. 7. In vitro readouts from the screening tool are protein-concentration dependent and correlate positively with clinical immunogenicity incidence. (A) (Left y-axis) Frequency of CXCR4⁺ moDC as a function of KLH concentration for one donor (mean±SEM). (Right y-axis) Transwell migration index as a function of KLH concentration in the lower chamber for two donors (mean±SEM). Treatments were tested in triplicate for each donor. (B) In vitro assay readouts versus the clinical ADA incidence for each therapeutic protein tested, labeled A-H. The y-axis represents the mean of three assay readouts in all donors (1-CXCR4 stimulation index, 2-CD40 stimulation index, and 3-Transwell migration index). Error bars are mean±SEM. The x-axis represents the highest reported clinical ADA incidence in US package inserts or literature. The best-fit line is shown for A-F (circles) with the 95% confidence interval. G and H (triangles) follow the data trend but fall outside of this 95% confidence interval. A—Anti-IL6R IgG, B—Bispecific IgG4, C—Anti-CD20 IgG, D—Anti-HER2 IgG, E—ATR-107, F—KLH, G—Anti-TNF IgG, and H—HuA33.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

The present disclosure provides a method for determining if a protein will produce an immunogenic response. The immunogenic response may be correlated by the migration of dendritic cells in the presence of the protein. The method may be used as a screening tool to assess immunogenicity risk for proteins that could be administered subcutaneously.

Without intending to be bound by any particular theory, it is considered that dendritic cell (DC) migration in the presence of a protein towards a chemokine ligand is indicative of an immunogenic response. Thus, in an embodiment, a method of the present disclosure may be used to determine if a therapeutic protein will induce an immunogenic response.

In various embodiments, a method of the present disclosure comprises utilizing a Transwell® plate (e.g., a plate comprising one or more Boyden chambers). Each Boyden chamber comprises a first chamber connected to and adjacent to (e.g., in fluid communication) with a second chamber, where the first chamber and second chamber are separated by a membrane (e.g., a semi-permeable or porous membrane). The pores of the membrane may be 5, 6, 7, 8, 9, or 10 μm. Cells only pass through the membrane via active migration.

A method of the present disclosure comprises determining migration of dendritic cells. A method may comprise contacting a known number of dendritic cells in the first chamber of the Boyden chamber with a therapeutic protein. The second chamber contains the therapeutic protein and one or more chemokines and does not contain any dendritic cells. Each chamber further comprises serum-free media. The concentration of the therapeutic protein in the first chamber and second chamber is different. The concentration of the therapeutic protein is lower in the first chamber than in the second chamber, such that a concentration gradient is formed between the two chambers. The chambers are incubated for a period of time. During incubation, some of the dendritic cells may migrate from the first chamber into the second chamber. The migrated dendritic cells may then be isolated, stained, and counted. Following counting, the amount of migrated dendritic cells may be used to determine the protein's immunogenicity response/risk. The immunogenicity response/risk may be determined by comparing the percent of dendritic cells migrated to a control.

The concentration of the therapeutic protein in the first chamber and second chamber is different. The concentration of the therapeutic protein is lower in the first chamber than in the second chamber. In various examples, the concentration of therapeutic protein in the second chamber is twofold, threefold, fourfold, fivefold, sixfold, sevenfold, eightfold, ninefold, or tenfold larger than in the first chamber. In various embodiments, the concentration of the therapeutic protein in the first chamber is 0 to 250 μg/mL, including all 0.1 μg/mL values and ranges therebetween (e.g., 25 to 250 μg/mL). In various embodiments, the concentration of the therapeutic protein in the first chamber is 100 μg/mL. In various embodiments, the concentration of therapeutic protein in the second chamber is 50 to 50,000 μg/mL, including all 0.1 μg/mL values and ranges therebetween. In various embodiments, the concentration of the therapeutic protein in the second chamber is 1000 μg/mL.

The second chamber may comprise two or more chemokines. In various examples, the second chamber comprises two chemokines. For example, the second chamber comprises CCL21 and CXCL12. The concentration of each chemokine is 1 to 200 ng/ml, including all 0.1 ng/mL values and ranges therebetween. In various embodiments, the concentration of each chemokine is 100 ng/mL. In various examples, the second chamber comprises three chemokines. For example, when there are three chemokines, the second chamber comprises CCL19, CCL21, and CXCL12. The concentration of each chemokine is 1 to 200 ng/mL, including all 0.1 ng/mL values and ranges therebetween. In various embodiments, the concentration of each chemokine is 100 ng/ml.

The first chamber comprises dendritic cells. Prior to migration the first chamber comprises 10,000 to 30,000 dendritic cells, including all integer values and ranges therebetween. In various examples, prior to migration, the first chamber comprises about 12,000=1000 dendritic cells.

The migration of may be stimulated by various therapeutic proteins. For example, the therapeutic proteins may be monoclonal antibodies. Additional examples of therapeutic proteins include, but are not limited to, recombinant enzymes, cytokines, globular proteins, AAV capsid proteins, antibody-drug conjugates, and the like, and combinations thereof.

In an aspect, the present disclosure provides a kit. The kit may provide instructions for use of the kit. The kit may further comprise media, one or more chemokines (e.g., CCL21 and CXCL12), and one or more plates comprising one or more Boyden chambers.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements are not intended to be limiting in any way.

Statement 1. A method for determining dendritic cell migration comprising: contacting a known amount of dendritic cells with a therapeutic protein at a first protein concentration in a first chamber, wherein the first chamber is connected to and adjacent to a second chamber and the first chamber and second chamber are separated by a porous membrane, wherein the second chamber contains the therapeutic protein at a second protein concentration and one or more chemokines and does not contain any dendritic cells; incubating the first chamber and second chamber; isolating migrated dendritic cells from the second chamber; staining the migrated dendritic cells from the second chamber; and counting the migrated dendritic cells from the second chamber, wherein the counted cells have migrated from the first chamber.

Statement 2. A method according to Statement 1, wherein the first protein concentration is lower than the second protein concentration.

Statement 3. A method according to Statement 1 or Statement 2, wherein the first protein concentration fivefold or tenfold less than the second protein concentration.

Statement 4. A method according to any one of the preceding Statements, wherein the first protein concentration is 0 to 250 μg/mL, including all μg/mL values and ranges therebetween.

Statement 5. A method according to Statement 4, wherein the first protein concentration is about or is 100 μg/mL.

Statement 6. A method according to any one of the preceding Statements, wherein the second protein concentration is 50 to 50,000 μg/mL, including all μg/mL values and ranges therebetween.

Statement 7. A method according to Statement 6, wherein the second protein concentration is about or is 1000 μg/mL.

Statement 8. A method according to any one of the preceding Statements, wherein the second chamber contains at least two chemokines.

Statement 9. A method according to Statement 8, wherein there are two chemokines, and the chemokines are CCL21 and CXCL12.

Statement 10. A method according to Statement 8, wherein there are three chemokines, and the chemokines are CCL19, CCL21, and CXCL12.

Statement 11. A method according to any one of Statements 8-10, wherein the concentration of each chemokine is 1 to 200 ng/mL, including all ng/mL values and ranges therebetween.

Statement 12. A method according to claim 11, wherein the concentration of each chemokine is about 100 ng/mL or is 100 ng/mL, including all ng/ml values and ranges therebetween.

Statement 13. A method according to any one of the preceding Statements, wherein prior to migration the first chamber comprises 10,000 to 30,000 dendritic cells, including all integer values and ranges therebetween.

Statement 14. A method according to Statement 13, wherein prior to migration the first chamber comprises about 12,000±1000 dendritic cells.

Statement 15. A method according to any one of the preceding Statements, wherein the therapeutic protein is suspected of inducing an immunogenic response in an individual.

Statement 16. A method according to any one of the preceding Statements, wherein the therapeutic protein is a monoclonal antibody.

Statement 17. A method according to any one of the preceding Statements, wherein the therapeutic protein is recombinant enzymes, cytokines, globular proteins, AAV capsid proteins, antibody-drug conjugtes, and the like, and combinations thereof.

Statement 18. A method according to any one of the preceding Statements, wherein the counting is performed by flow cytometry.

Statement 19. A method according to any one of the preceding Statements, wherein the porous membrane has pore size of 5 to 10 μm (e.g., 5 or 8 μm), including all μm values and ranges therebetween.

Statement 20. A method according to any one of the preceding Statements, further comprising determining the percent of dendritic cells that migrated.

Statement 21. A method according to any one of the preceding Statements, further comprises determining a migration index by comparing the percent of migrated cells to a percent of migrated cells from a control group.

Statement 22. A method according to any one of the preceding Statements, wherein the method utilizes a multiwelled (e.g., Transwell®) plate comprising one or more Boyden chambers.

The following examples are presented to illustrate the present disclosure. They are not intended to be limiting in any matter.

### Example 1

This example provides a description of a method of the present disclosure.

The efficacy and safety of therapeutic proteins are undermined by immunogenicity driven by anti-drug antibodies (ADA). Proteins administered subcutaneously can suffer from enhanced immunogenic potential compared to intravenous administration. Immunogenicity risk assessment is critically necessary during drug development, but current methods lack predictive power and mechanistic insight into the subcutaneous immune response. The migration of cutaneous dendritic cells (DC) into the injection site and toward draining lymph nodes for T-cell activation is proposed to drive subcutaneous immunogenicity. Thus, the migratory potential of DC was identified as a novel mechanistic marker for immunogenicity screening. Immunogenic risk was signaled by an increased ability of the therapeutic protein to induce DC migration along a chemokine gradient. Also, expression of the chemokine receptor CXCR4 on human monocyte-derived DC, in combination with activation markers CD40 and IL-12, strongly correlated with clinical ADA incidence. Application of this mechanism-based screening tool will improve immunogenicity risk assessment for subcutaneously administered therapeutic proteins, and this technology could be adapted for immunogenicity prediction of subunit vaccine candidates, gene therapy vectors, and other biological modalities.

SC injection introduces a high concentration of therapeutic protein into the skin, a site well-known for immune reactions mediated by epidermal Langerhans cells and dermal dendritic cells. Dermal DCs are continuously surveying the skin for antigens to capture and bring to nearby draining lymph nodes (DLN) to then induce T cell activation, a process which occurs at homeostasis and becomes upregulated during inflammation. Migration of DCs is a multi-phasic process that includes migration of immature DC from the epidermis or dermis into the hypodermis, migration of semi-mature/mature DC from the skin into the initial lymphatics, transport to the subcapsular sinus of a draining lymph node, and migration into the paracortex or T-cell area. The major driver of all migration stages is the receptor-ligand interaction between CCR7 on DCs and its ligands CCL19 and CCL21. Expression of CCL21 on the lymphatic endothelium and on fibroblastic reticular cells in the DLN create a haptotactic gradient for DC migration. Furthermore, CXCR4 expression on mature cutaneous DCs and the interaction with ligand CXCL12 plays a major role in their migration through the skin and into the lymphatics. CD4⁺ T cell activation is strongly induced by antigen loaded migratory DCs arriving in the lymph node, which have matured along the migration path. The degree of DC maturation and migration is a function of the context of antigen exposure (e.g., inflammation) and the features of the antigen. It is hypothesized that a therapeutic protein would upregulate DC migration into the SC injection site and toward the DLNs in proportion to its immunogenic potential. Furthermore, the presence of risk factors (danger signals or adjuvants) in the drug product or injection site could increase skin-derived DC migration. By exposing dendritic cells to therapeutic protein in vitro, the induction of migratory potential could be assessed and transformed into a marker for immunogenic risk.

A comprehensive in vitro immunogenicity prediction tool was generated for subcutaneously administered therapeutic proteins. Migratory potential of DCs in response to therapeutic protein was captured by CXCR4 expression and by migration toward associated chemokine ligands; and these readouts correlated with and predicted immunogenic risk of multiple protein antigens. Also, since DC migration can occur under tolerogenic or homeostatic conditions, subsequent DC maturation status was assessed using key activation markers CD40 and IL-12. Combining DC activation markers with migratory potential improved predictive power and produced a total stimulation index readout that correlated strongly with clinical immunogenicity incidence for a range of therapeutic proteins.

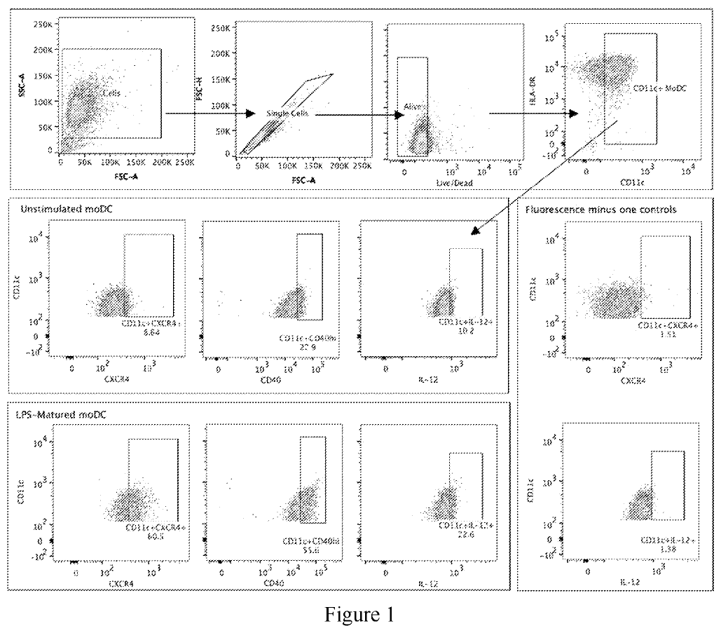

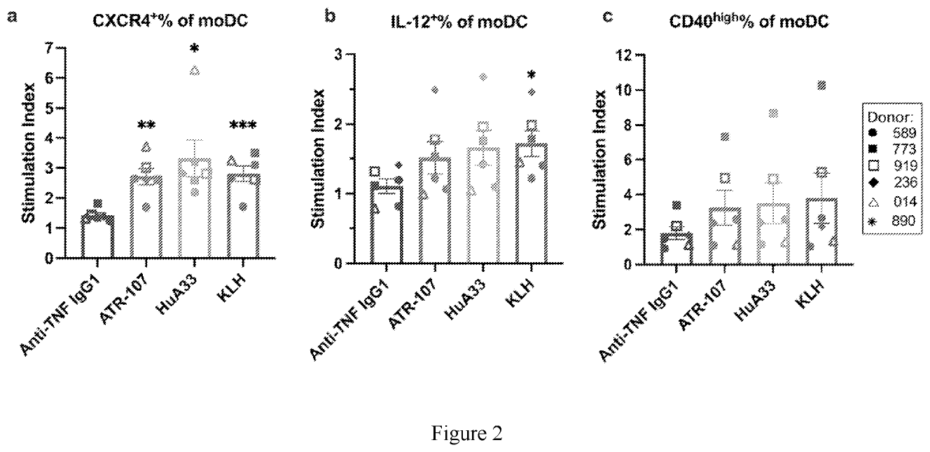

CXCR4 is upregulated on dendritic cells by immunogenic therapeutic proteins. It was sought to determine if CXCR4 was upregulated on dendritic cells by therapeutic proteins in proportion to their risk of immunogenicity. Monocyte-derived dendritic cells (moDC) from six healthy donors were stimulated with therapeutic proteins for 24 hours followed by flow cytometry analysis for CXCR4 expression (FIG. 1). Keyhole limpet hemocyanin (KLH), a highly immunogenic protein, was used as the positive control, which is common for in vitro immunogenicity prediction assays. The ability of three therapeutic proteins to upregulate CXCR4 was tested. Anti-TNF IgG1 is a monoclonal antibody with varying ADA incidence across patient populations ranging from 3-28% (low-moderate), and HuA33 and ATR-107 are monoclonal antibodies with high ADA incidence in phase I clinical trials (73 and 76%, respectively). It is believed that therapeutic proteins with immunogenic potential will upregulate CXCR4 concurrently with DC activation and maturation. The average frequency of CXCR4⁺ moDC in six donors was upregulated from 5.1±1.2% when unstimulated to 7.8±1.4%, 13.1±3.0%, 16.2±3.7%, and 14.4±3.9% in response to Anti-TNF IgG1, ATR-107, HuA33, and KLH, respectively (mean±SEM). The stimulation index (S.I.) was calculated by dividing the frequency of CXCR4⁺ (%) moDC in each treatment group by the average frequency of CXCR4⁺ (%) moDC in the unstimulated group. The stimulation index of ATR-107 and HuA33 in a population of six donors was significantly upregulated (p=0.001 and 0.0117, respectively) compared to anti-TNF IgG1, similar to the increase seen with KLH (p=0.0005, unpaired student t-test) (FIG. 2a). Furthermore, the upregulation of CXCR4 was found to be dose dependent. The frequency of CXCR4⁺ moDC increased with increasing concentration of KLH reaching 64.4% at 500 μg/ml. But in the presence of the activation signal, a dose of 5 μg/ml KLH was sufficient to induce a significant increase in CXCR4 expression over the unstimulated control (p=0.0147).

CXCR4 upregulation correlates with maturation/activation marker upregulation by immunogenic therapeutic proteins. Upon maturation in the skin, dermal DCs and Langerhans cells, especially, will upregulate CXCR4 to migrate toward the lymphatics and draining lymph nodes. Chemokine-directed migration is also a phenomenon of immature or semi-mature DCs, thus maturation status will be assessed in combination with migratory potential. We sought to confirm that therapeutic proteins with immunogenic risk upregulate DC maturation markers concurrently with CXCR4. The two markers selected, CD40 and IL-12, will provide the activation status of DCs. In response to treatment with ATR-107 and HuA33, an increase in the frequency of IL-12-producing (IL-12⁺) DCs was observed by flow cytometry. The observed stimulation index was 1.51±0.23 for ATR-107 and 1.66±0.25 for HuA33 in a panel of six donors, which was comparable to that of KLH (1.72±0.19) (mean±SEM) (FIG. 2b). Anti-TNF IgG1 only slightly upregulated IL-12⁺ moDC over baseline, with an average stimulation index of 1.11±0.10, which was significantly less than KLH (p=0.0171). The frequency of CD40^{high} moDCs was also upregulated by KLH, ATR-107, and HuA33, indicated by an average stimulation index of 3.80±1.43, 3.25±0.99, and 3.50±1.17, respectively (mean±SEM) (FIG. 2c). Thus, compared to that of anti-TNF IgG1 (S.I.=1.80±0.36), the stimulation index for therapeutic proteins with immunogenic risk was almost 2-fold higher (ns). Concurrent upregulation of migration (CXCR4) and activation (IL-12, CD40) markers on dendritic cells can capture the intrinsic immunogenic potential of therapeutic protein.

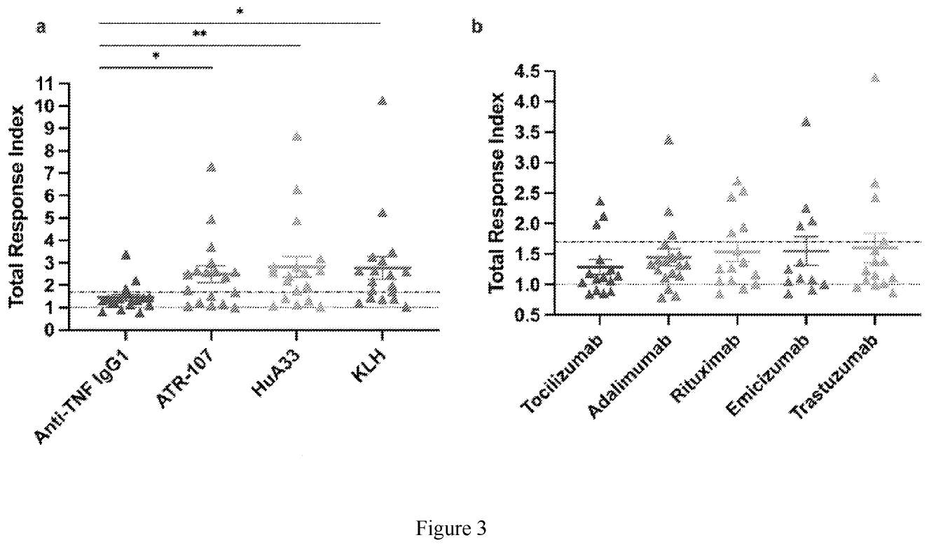

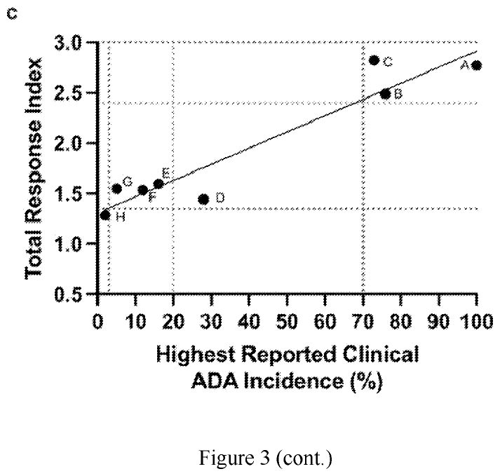

The combined readout of the screening assay correlates with immunogenicity incidence for a panel of therapeutic proteins. To validate these immunogenicity risk assessment markers, a panel of protein antigens was assembled, including KLH, ATR-107, HuA33, adalimumab, tocilizumab, trastuzumab, rituximab, and emicizumab. Clinical immunogenicity incidence upon SC administration is available for all these therapeutic proteins, except HuA33 (Table 1). MoDC were exposed to 5 μg/ml of therapeutic protein in the presence of the activation signal for 24 h followed by flow cytometry analysis. The stimulation index for each marker was calculated by dividing the frequency of CXCR4⁺, IL-12⁺, and CD40^{high} moDC by the corresponding average frequency in unstimulated moDC. Then the mean of all stimulation index values observed in the panel of healthy donors was denoted the ‘Total Response Index’ (TRI). This index will allow the ranking of each protein as having low, moderate, or high immunogenicity risk following SC administration. The TRI for highly immunogenic proteins, KLH, ATR-107, and HuA33, was in the range of 2.4 to 2.8, while a range of 1.4 to 1.6 was observed for proteins with low to moderate immunogenicity incidence, namely, adalimumab, rituximab, trastuzumab, and emicizumab (FIG. 3a-b, Table 1). Then the monoclonal antibody with the lowest clinical immunogenicity incidence, tocilizumab, had a TRI of 1.3. By plotting TRI versus the highest reported immunogenicity incidence in clinical trials, a positive correlation is observed, and proteins can be grouped into very low, low to moderate, and high immunogenic potential (FIG. 3c).

TABLE 1
The clinically reported anti-drug antibody incidence for therapeutic proteins and their
in vitro screening tool readout (Total Response Index) for immunogenicity risk.

| | Total Response | | | | ADA |
|---|---|---|---|---|---|
| | Index from in | Type of | Molecular | Route of | Incidence |
| | vitro assay* | Molecule | Target | Administration | (%) |
| Tocilizumab | 1.29 ± 0.13 (N = 15) | Humanized | IL-6R | Subcutaneous | 0.8-2% |
| | | IgG1 | | | |
| Rituximab | 1.54 ± 0.16 (N = 15) | Chimeric | CD20 | Subcutaneous | 2-12% |
| | | murine/human | | | |
| | | IgG1 | | | |
| Adalimumab | 1.44 ± 0.14 (N = 18) | Human IgG1 | TNFa | Subcutaneous | 3-28% |
| Emicizumab | 1.55 ± 0.24 (N = 12) | Humanized | FX and | Subcutaneous | 5.10% |
| | | IgG4 | FIXa | | |
| Trastuzumab | 1.60 ± 0.24 (N = 15) | Humanized | HER2 | Subcutaneous | 16% |
| | | IgG1 | | | |
| ATR-107 | 2.49 ± 0.37 (N = 18) | Human IgG1 | IL-21R | Subcutaneous/ | 76% |
| | | | | Intravenous | |
| HuA33 | 2.83 ± 0.47 (N = 18) | Humanized | A33 | Intravenous | 73% |
| | | IgG1 | | | |

*Mean ± SEM, N = number of responses. ADA Anti-drug antibody, USPI United States Package Insert.

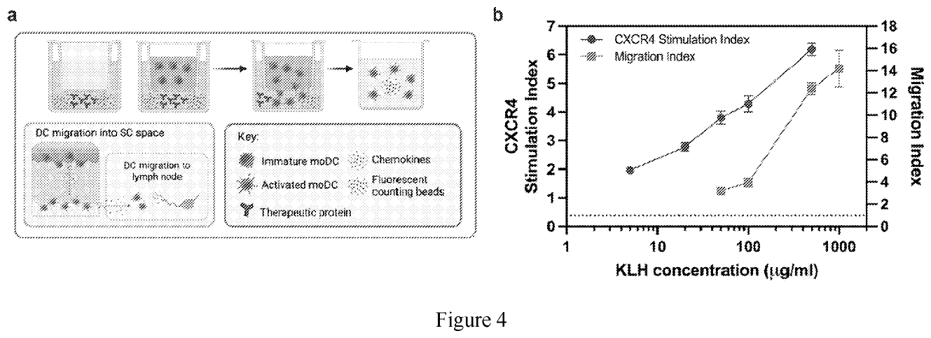

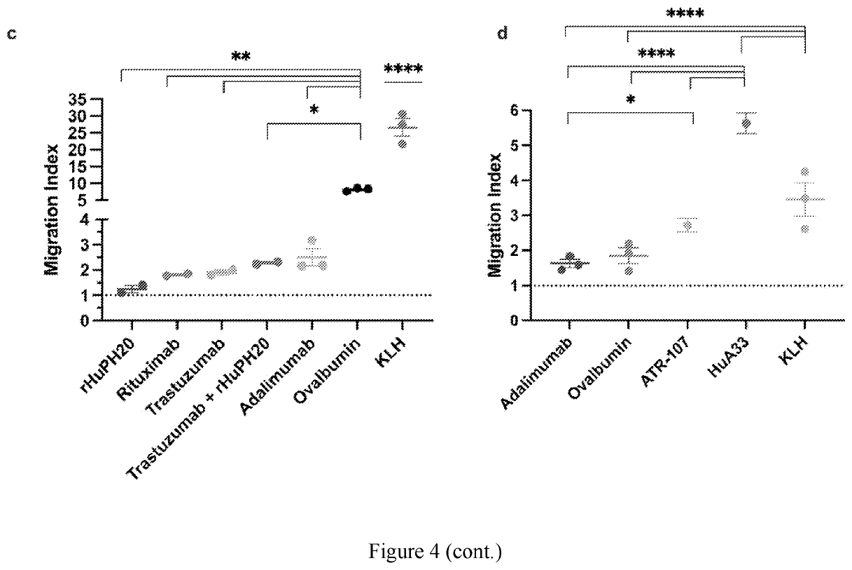

Immunogenic therapeutic proteins not only upregulate CXCR4 but directly stimulate migration of moDC toward chemokines. Following antigen uptake and processing, moDCs upregulate chemokine receptors, costimulatory molecules, and proinflammatory cytokines, establishing a mature, migratory phenotype that drives strong CD4⁺ T-cell activation in DLNs. However, the likelihood of dendritic cell migration into the injection site is not entirely captured by these phenotypic markers. In this case, migration potential toward therapeutic protein should be directly captured in vitro. Thus, a Transwell assay was designed to test whether immunogenic proteins drive DC migration in the presence of chemokine ligands (FIG. 4a). In preliminary experiments, DC migration toward a combined gradient of two chemokine ligands was superior to a gradient of either chemokine alone (data not shown). When 50 μg/ml KLH was added to the bottom chamber of Transwell inserts in addition to chemokine ligands, immature moDC migrated 3.5-fold more than toward the chemokines alone. The increase in moDC migration induced by the protein antigen, expressed as Migration Index, correlated with the CXCR4 stimulation index (FIG. 4b). KLH and ovalbumin induced a migration index of approximately 26-fold and 8-fold, respectively, in three donors (FIG. 4c). But at the same concentration, proteins with low to moderate immunogenic risk, namely adalimumab, rituximab, and trastuzumab, do not induce migration as strongly indicated by a migration index between 2 and 2.5. Additionally, creating a protein concentration gradient 5-fold lower in magnitude was sufficient to capture the high immunogenic risk of ATR-107 and HuA33 (FIG. 4d).

Immunogenicity can undermine the clinical utility of therapeutic proteins by introducing safety concerns and impacting clinical response. Unique immunogenicity challenges are introduced by the subcutaneous route of administration for biologics compared to other delivery routes. It is imperative to perform immunogenicity risk assessment during development stages, but predictive power and mechanistic insight are lacking for many available methods. In this novel screening tool, the migratory potential of dendritic cells was transformed into a marker for immunogenic risk since dendritic cell migration is proposed to drive subcutaneous immunogenicity. DCs expressing CXCR4 and CD40 in addition to producing IL-12 will be strong inducers of CD4⁺ T cell activation and Th1 cell differentiation. By combining migratory potential with the activation phenotype markers, CD40 and IL-12, a robust assay readout was generated to predict immunogenic risk for a panel of therapeutic proteins.

The readout from this in vitro screening tool can predict immunogenicity risk of several therapeutic proteins, including two highly immunogenic mAbs, ATR-107 and HuA33. ATR-107 was found to upregulate CXCR4, IL-12, and CD40 on dendritic cells, which correlates with results known in the art. Investigation into the molecular mechanisms of ATR-107 immunogenicity found that it induced expression of DC maturation markers, such as CD86 and CD40, significantly more than a control antibody. Furthermore, a population of CCR7-expressing DCs was upregulated in the presence of ATR-107, corresponding to our findings that ATR-107 can induce moDC migration toward the chemokine ligands in the Transwell assay. Similar outcomes were observed for the immunogenic mAb HuA33 in our screening assay. Because the dendritic cell markers were most successful at predicting a high risk of immunogenicity, it is highly likely that this screening tool would be able to screen immunogenicity of subunit vaccine candidates.

The combined assay readout, including responses to all phenotypic markers, was found to correlate positively with the highest reported clinical incidence of ADA. Predicting the risk of immunogenic response, by determining the severity of the event and/or the probability of occurrence, is the goal for an in vitro screening tool. Here, the Total Response Index was used as an output for the severity of risk, and the number of positive responders as an assay readout was not reported. Although the donor pool used here was sufficient to capture some inter-individual variability, a much larger donor pool would be required to attempt to predict the number of subjects likely to develop ADA. The screening tool could be further validated by including mAbs that share a target but differ in immunogenicity incidence, for example anti-PCSK9 antibodies bococizumab, alirocumab, and evolocumab, or anti-IL-17A antibodies ixekizumab and secukinumab. Bococizumab induces T-cell activation and DC maturation in vitro, thus we would expect bococizumab to upregulate CXCR4 concurrently with CD40 and IL-12 leading to increased DC migration into and out of the subcutaneous injection site. If CXCR4 and Transwell DC migration were not upregulated by other anti-PCSK9 mAbs, alirocumab and/or evolocumab, this would indicate a unique mechanism by which bococizumab could induce a stronger and neutralizing immune response.

CXCR4 has not been considered as a marker for immunogenic risk; however, we found it to be reliably upregulated in proportion to a protein's immunogenic potential. Furthermore, the upregulation of CXCR4 corresponded to an increase in DC migration toward a combination of chemokine ligands. The CXCR4⁺ moDCs are a robust cell population that can be easily identified by flow cytometry analysis when proper controls are utilized for gating. In comparison, other immunogenicity risk assessment strategies rely on detection of antigen-specific T helper cells, where the population frequency is as small as 0.1%, which could cause convoluted flow cytometry analysis. The moDCs used in this in vitro screening tool are equivalent or similar in phenotype to those used in other immunogenicity risk assessment strategies, such as MAPPS and DC internalization assays. Furthermore, these cells express similar phenotypic characteristics to dermal dendritic cell populations, indicating their suitability for our assay. The Transwell migration assay is a powerful tool for testing DC migration in a short amount of time with few cells (in the 96-well format), and hands-free analysis is made possible by the flow cytometer autosampler function. The incorporation of this method with other in vitro screening assays would be quite straightforward with flow cytometry being commonly employed in immunogenicity assessment.

By capturing the likelihood of DC migration from the epidermis/dermis to the hypodermis, a Transwell assay can be used to test for the impact of therapeutic protein characteristics on immunogenic risk following subcutaneous administration. The Transwell migration index even demonstrated better predictive power for some mAbs, like adalimumab, for which the Total Response Index did not accurately predict its immunogenic potential. Also, DC migration was found to be concentration dependent. Injection site concentrations of mAb are likely sufficient to induce strong DC migration from the upper layers of skin, according to the immunogenic potential of the mAb, since high concentrations of protein, e.g., 100 mg/ml, are typically formulated for subcutaneous administration. Product characteristics can also impact the likelihood of DC migration, such as protein structural features, instability pathways (e.g., aggregates), or formulation properties (e.g., viscosity) that prolong injection site retention time. Many product-related risk factors could be screened in the Transwell migration assay, and the ability of the assay to predict the impact of stressed protein molecules on moDC migration is currently under investigation. In an in vitro skin model, aggregated mAbs were found to induce proinflammatory cytokine and chemokine production by skin cells, suggesting a mechanism by which aggregates could increase DC migration into the SC injection site. The injection of hyaluronidase with highly concentrated mAbs can improve absorption time and bioavailability; however, the immunogenicity of the co-administered mAb is not reduced. Furthermore, it was found that hyaluronidase increased migration of moDC toward trastuzumab for two donors, although a statistically significant difference was not achieved.

It is hypothesized that the applicability of this in vitro testing system for immunogenicity extends beyond proteins and toward novel biological modalities, in addition to recognizing risk introduced by changes in formulation, protein structure/post-translational modifications, concentration, and more. Novel therapeutic modalities, like AAV vectors, CAR T cells, nucleic acids, and so on, have unique immunogenicity concerns and appear to differ in immune activation mechanisms compared to the therapeutic proteins. But because dendritic cells are extensively involved in immune responses toward all types of antigens, they remain a feasible cellular option for in vitro screening of novel modalities. Introducing mechanism-based markers into immunogenicity risk assessment of biologics should improve predictive power and the understanding of risk factors behind immunogenicity.

Heat inactivated male type AB human serum, gentamicin sulfate, penicillin/streptomycin 100× solution, and keyhole limpet hemocyanin (KLH) were purchased from Sigma-Aldrich (St. Louis, MO). Fluorescently labeled antibodies, compensation beads, cell permeabilization buffer, brefeldin A, monensin, and viability ghost dye for flow cytometry were purchased from Tonbo™—a Cytek® Brand and Biolegend (San Diego, CA). CountBright™ Absolute Counting Beads were obtained from Thermo Fisher Scientific (Waltham, MA). Human cytokines (IL-4, GM-CSF) and chemokines were purchased from Sino Biological (Wayne, PA). Corning Inc. (Corning, NY) RPMI-1640, cell culture plates, and Transwell plates (3384, 3387) were used for all experiments. Biosimilar research-grade monoclonal antibodies were purchased from Absolute Antibody (Boston, MA) (tocilizumab, ATR-107, and HuA33) and Bio X Cell (Lebanon, NH) (adalimumab, trastuzumab, and rituximab). Emicizumab (Hemlibra) was generously provided by WNY BloodCare (Buffalo, NY). Endo Grade ovalbumin was purchased from BioVendor (Asheville, NC).

Culture of human monocyte-derived dendritic cells. Cryopreserved healthy HLA-typed human peripheral blood mononuclear cells (PBMC) were purchased from Cytologics (San Diego, CA) and stored in vapor phase liquid nitrogen until use. Donor information is provided in Supplementary Table 1. Classical CD14⁺CD16⁻ monocytes were isolated by negative selection on Miltenyi MS columns following the instructions provided in the Classical Monocyte isolation kit (Miltenyi Biotec, Gaithersburg, MD). Monocytes were cultured at 37° C. and 5% CO₂ for five days in complete media at approximately 5×10⁵ cells/ml with 50 ng/ml IL-4 and 50 ng/ml GM-CSF. Complete media was RPMI-1640 with 10% human serum, 1% Penicillin/streptomycin, 30 μg/ml gentamicin sulfate, and 50 M 2-mercaptoethanol. Half of the media was changed every two days and replaced with fresh media containing 100 ng/ml IL-4 and 100 ng/ml GM-CSF. On day five, moDCs were harvested and used immediately in immunogenicity screening experiments. DC differentiation was checked by flow cytometry (CD11c⁺HLA-DR⁺DC-SIGN⁺CD14^{low})

TABLE 1
Characteristics of healthy donor sources for peripheral blood mononuclear
cells. Donor information was provided by the vendor Cytologics LLC.

| Donor | Age | Gender | Race | BMI | HLA-A | | HLA-B | | HLA-C | |
|---|---|---|---|---|---|---|---|---|---|---|
| 589 | 27 | Male | White | 26.9 | 02:01 | 26:08 | 07:02 | 13:02 | 06:02 | 07:02 |
| 919 | 36 | Male | White | 31.2 | 01:01 | 26:01 | 08:01 | 38:01 | 07:01/02 | 12:02/03 |
| 236 | 46 | Male | White | 32.1 | 01:01 | 01:01 | 08:01 | 51:01 | 07:01 | 16:01/02 |
| 773 | 40 | Male | White | 28.9 | 11:01 | 11:01 | 15:01 | 49:01 | 03:02/04/05 | 07:01/02/05 |
| 890 | 27 | Male | White | 31.9 | 02:01 | 03:01 | 15:01 | 40:01 | 03:02/04/05/06 | 03:04/06/09 |
| 014 | 24 | Female | White | 28.9 | 02:01 | 29:01/02 | 44:03 | 51:01 | 15:02/03/07 | 16:01/02/08 |

BMI Body mass index, HLA Human leukocyte antigen.

Dendritic cell markers for immunogenicity prediction. Immature moDC were cultured at 37° C. and 5% CO₂ for 24 hours in complete media with 5 μg/ml therapeutic protein in the presence of 100 μg/ml lipopolysaccharide (LPS) as an activation signal. Control conditions included unstimulated moDC cultured in media alone and mature moDC cultured with 1 μg/ml LPS. To improve the sensitivity of intracellular staining, protein transport inhibitors brefeldin A and monensin were added five hours before harvesting. MoDC were stained for CD11c (FITC or PerCP-Cy5.5), HLA-DR (APC-Cy7), CD40 (PE), CXCR4 (APC), and live/dead (Violet450 ghost dye). Then cells were fixed in 2% buffered formalin and permeabilized for intracellular staining of IL-12 (PerCP-Cy5.5 or FITC). Stained moDC were stored in PBS at 4° C. and analyzed on the BD LSRFortessa. Unstained, single stain, and fluorescence-minus-one samples were prepared to facilitate compensation and gating strategies. The main cell population was gated by FSC vs SSC, then single cells were gated by FSC-H vs FSC-A, and live cells were gated by low viability dye expression (FIG. 1). MoDC were gated as CD11c⁺HLA-DR⁺ and represented the majority (≥75%) of live cells present. MoDC were then gated to determine the following population frequencies: CXCR4⁺, IL-12⁺, and CD40^{high}.

Dendritic cell migration toward therapeutic protein. Migration of immature moDC toward therapeutic protein was tested in a Transwell assay by creating a concentration gradient of therapeutic protein and chemokines across the insert. Therapeutic protein formulations were prepared in media with two chemokine ligands and added to the bottom chambers of a 96-well Transwell plate. Control conditions included media with and without chemokines. The upper chambers were filled with immature moDC in media containing therapeutic protein at a concentration 5- or 10-fold lower than that in the lower chambers. Transwell plates were incubated at 37° C. and 5% CO₂ to allow migration of moDC into the bottom chamber. Then the Transwell insert tray was removed and media was aspirated from the upper chambers which were then filled with washing buffer. The Transwell plate was centrifuged and supernatant discarded then wells were filled with washing buffer. The insert tray was replaced and incubated at room temperature for 10 minutes to allow dissociation of cells attached to the underside of the insert. The insert tray was then discarded. After a second centrifuge step, cells were stained with DC-SIGN PE in MACS buffer for 30 minutes. Following a final centrifugation step, cells were fixed in 2% buffered formalin and CountBright counting beads were spiked into each well to facilitate accurate cell counting. Migrated moDC were counted in the Transwell plate on a Miltenyi MACSQuant 10 flow cytometer using the autosampler function. Percent migrated was determined as (number of migrated moDC/number of plated moDC)*100%.

Statistical analysis. Flow cytometry data was analyzed in FlowJo v10.7. All statistical analysis was performed in GraphPad Prism v9. Statistical significance of flow cytometry results was determined by unpaired student's t-test or one-way ANOVA with Tukey's or Dunnett's multiple comparisons test at significance level alpha=0.05.

### Example 2

This example provides a description of a method of the present disclosure.

Transwell assay for immunogenicity screening
Corning 96-well Transwell plate with 5 or 8 μm pore size
Lower chambers (LC) receive 100 ng/ml CCL21 and 100 ng/ml CXCL12, in addition to 1000 μg/ml therapeutic proteins, in 235 μL per well.
Upper chambers (UC) receive 12,000 immature DCs and 100 μg/ml therapeutic protein in 75 μL per well.
Control groups include:
   Blank (UC: Cells in Media, LC: Media)
   Media (UC: Cells in Media, LC: Chemokines)
   KLH (UC: KLH 100 μg/ml+Cells in Media, LC: KLH 1000 ug/ml+Chemokines)
Incubation time is 2.5 hours.
All media (RPMI-1640) during Transwell test is serum-free.

Part 1: Harvest and count IMMATURE DCS
1. Mix cells well in plate and transfer to 50 ml tube.
2. Add 1 ml cold PBS to each well and scrape bottom of well gently with 1 ml syringe plungers, mix well and transfer to tube.
3. Centrifuge 500×g 5 min then remove supernatant and resuspend in 10 ml RPMI.
4. Centrifuge again then remove supernatant and resuspend in 1-3 ml RPMI.
5. Take 20 μL aliquot and perform a cell count.

Part 2: Prepare a solution of 10 μg/ml CCL21 and CXCL12
6. Prepare chemokines at 10 μg/ml then add to protein formulations to dilute to 100 ng/ml (10/0.1=100-fold).
7. To prepare solution if stock concentration of each chemokine is 250 μg/ml:

$$a . 8 μL CCL 21 + 8 μL CXCL 12 + 184 μL media = 200 μL total$$

8. To each 750 μL formulation, add 7.5 μl of 10 μg/ml chemokine stock.

$$b . ( 750 μL * 100 ng / ml ) / 10000 ng / ml = 7.5 μL$$

Part 3: Prepare formulations containing protein and chemokines for lower chambers:
9. Each lower chamber needs 235 μL, thus in triplicate, at least 705 μL is needed.
10. Add volume of media to each tube, then add proteins, and finally add chemokines just before plating.
11. Mix formulations well.

| | # of | | Stock | Dose | V final | | V initial | Chemokines | V media |
|---|---|---|---|---|---|---|---|---|---|
| | wells | Treatment | mg/ml | mg/ml | (μL) | DF | (μL) | (μL) | (μL) |
| 1 | 3 | BLANK | | | 750 | | 0 | 0 | 750 |
| 2 | 3 | MEDIA | | | 750 | | 0 | 7.5 | 742.5 |
| 3 | 3 | KLH | 10 | 1 | 750 | 10 | 75 | 7.5 | 667.5 |
| 4 | 3 | Protein 1 | | 1 | 750 | | | 7.5 | |
| 5 | 3 | Protein 2 | | 1 | 750 | | | 7.5 | |
| . . . | . . . | . . . | | | | | | | |

Part 4: Prepare formulations containing protein and immature DC for upper chambers:
12. Each upper chamber needs 75 μL, thus in triplicate, at least 225 μL is needed.
13. Prepare cells at [12,000 cells/0.075 ml=160,000 cells/ml] for plating.
14. If cell concentration was 2×10⁶ cells/ml from step 5, then:

$$a . ( 2 \times {10}^{6} cells / ml ) * V 1 = ( 0.16 \times {10}^{6} cells / ml ) * ( 250 μL ) , V 1 = 20 μL cells$$

15. Add volume of media to each tube, add appropriate volume of cells, and then add proteins.
16. Mix formulations well.

| | # of | | Stock | Dose | V final | | V initial | V cells | V media |
|---|---|---|---|---|---|---|---|---|---|
| | wells | Treatment | mg/ml | mg/ml | (μL) | DF | (μL) | (μL) | (μL) |
| 1 | 3 | BLANK | — | — | 250 | — | | 20 | 230 |
| 2 | 3 | MEDIA | — | — | 250 | — | | 20 | 230 |
| 3 | 3 | KLH | 10 | 0.1 | 250 | 100 | 2.5 | 20 | 227.5 |
| 4 | 3 | Protein 1 | | 0.1 | 250 | | | 20 | |
| 5 | 3 | Protein 2 | | 0.1 | 250 | | | 20 | |

Part 5: Set up Transwell plate
17. Label the Transwell plate lid.
18. Lift the insert tray and store in a sterile 96-well plate.
19. Plate 235 μL in triplicate of each formulation containing protein and chemokines in the bottom chambers.
20. Replace insert tray.
21. Plate 75 μL in triplicate of each formulation containing cells and protein in the upper chambers.
22. Cover and incubate at 37° C., 5% CO₂ for 2.5 hours.

Part 6: Collect migrated cells and prepare for counting
1. Prepare washing buffer: 10 mM EDTA in HBSS.

$$a . For 50 ml = 1 ml EDTA + 49 ml HBSS$$

2. Prepare staining buffer: 2 mM EDTA, 0.5% FBS in PBS.

$$a . For 50 ml = 0.2 ml EDTA + 0.25 ml FBS + 49.55 ml PBS$$

3. Remove insert tray and tap out medium from the inside of the Transwell inserts onto a pad.
4. Place tray in a clean 96-well plate. Add 75 μL of washing buffer to each well.
5. Centrifuge plate (PlateFuge 600×g 5 min) and decant supernatant then tap on pad to remove excess.
6. Add 200 μL washing buffer to all wells and replace the insert tray.
7. Incubate for 10 min at room temperature to detach cells on bottom of insert. Tap the Transwell plate gently against a hard surface to dislodge any remaining cells.
8. Remove and discard insert tray. Centrifuge plate and decant supernatant then tap on pad to remove excess.
9. Add 200 μL staining buffer to all wells and mix well by pipetting up and down.
10. Dilute 50 μL fluorescently-labeled DC marker antibody with 2.950 ml staining buffer and add 30 μL/well using a repeater pipette. Mix well and incubate 30 min in the fridge.
   a. Such as, CD11c AlexaFluor700 or DC-SIGN PE
11. Centrifuge plate and decant supernatant then tap on pad to remove excess.
12. Add 300 μL 2% buffered formalin to all wells and mix well by pipetting up and down.
13. Record the exact volume in a well.
14. Store plate covered in fridge until analysis.
15. Immediately before flow cytometry analysis spike in fluorescent counting beads.
   a. Vortex beads for 30 seconds first.
   b. Dilute beads 25-fold to 40 beads/ul from stock (50000 beads/50 μL=1000 beads/μL) then add 30 μL/well for 1200 beads/well.
   c. For whole plate:

$$i . 30 μL * 100 = 3000 μL \to 3000 μL / 25 = 120 μL beads \to 120 μL beads + 2.88 μL MACS buffer$$

d. Vortex well to mix, then transfer to trough and use multichannel pipette to add 30 μL/well.

Part 7: Flow cytometry analysis
1. Analyze the wells on the Miltenyi MACSQuant 10 using the Chill Rack 96-well autosampler function. Run on HIGH flow rate with FAST washing mode.
2. Adjust the voltage settings so the beads and cell populations both show up on the plot.
3. For each well, analyze at least 100 μL and collect the bead and cell events.

$$4. Calculate the cell concentration as : ( Cell events / Bead events ) * ( \# of Beads added / Sample volume )$$

$$5. Calculate the number of cells migrated as : Cell concentration * Sample \\ volume$$

$$6. Calculate the percent migrated as : ( Number of cells migrated / Number of cells plated in the upper chamber ) * 100 \%$$

$$7. Calculate the Migration Index as : ( Percent migrated / Average percent migrated in ‘ Media ’ group )$$

### Example 3

This example provides a description of a method of the present disclosure.

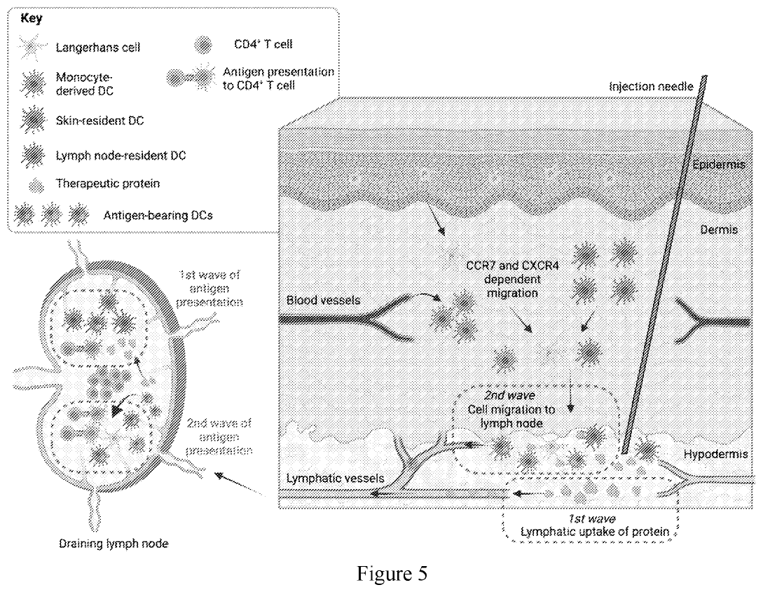

The safety and efficacy of therapeutic proteins are undermined by immunogenicity or unwanted immune response driven by anti-drug antibodies (ADA). In particular, proteins injected subcutaneously can suffer from enhanced immunogenic potential compared to intravenous delivery. However, there is a lack of immunogenicity risk assessment methods designed for the prediction of immunogenic potential by subcutaneous (SC) delivery. SC injection introduces a high concentration of therapeutic protein into the skin where there are many skin-resident dendritic cells continuously surveying for antigens. Following capture of protein antigens, migration of these skin-resident dendritic cells from the injection site to draining lymph nodes drives cognate T-cell activation and subsequent humoral responses (FIG. 5).

The ability of a therapeutic protein to induce dendritic cell (DC) migration is proposed to predict its immunogenic potential following SC injection. Here, DC migratory potential was investigated as a novel marker for immunogenicity screening.

### Methods.

1. Healthy HLA-typed PBMCs were purchased from Cytologics LLC, and six donors were included to account for inter-individual variability in immunogenicity.

2. Classical CD14⁺CD16⁻ monocytes were isolated from PBMCs by negative selection on a Miltenyi magnetic column.

3. Monocytes were differentiated into HLA-DR⁺CD11c⁺DC-SIGN⁺ monocyte-derived DCs (moDC) by culturing with IL-4 and GM-CSF.

4. Immature moDC were harvested on day five and plated with therapeutic proteins in media overnight. Control treatment groups were unstimulated/media, lipopolysaccharide (LPS), and keyhole limpet hemocyanin (KLH).

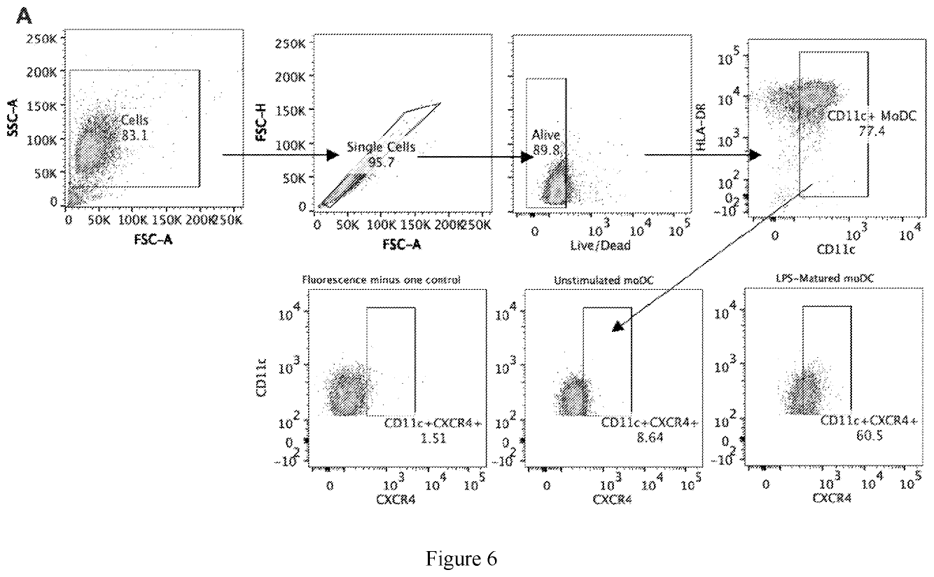

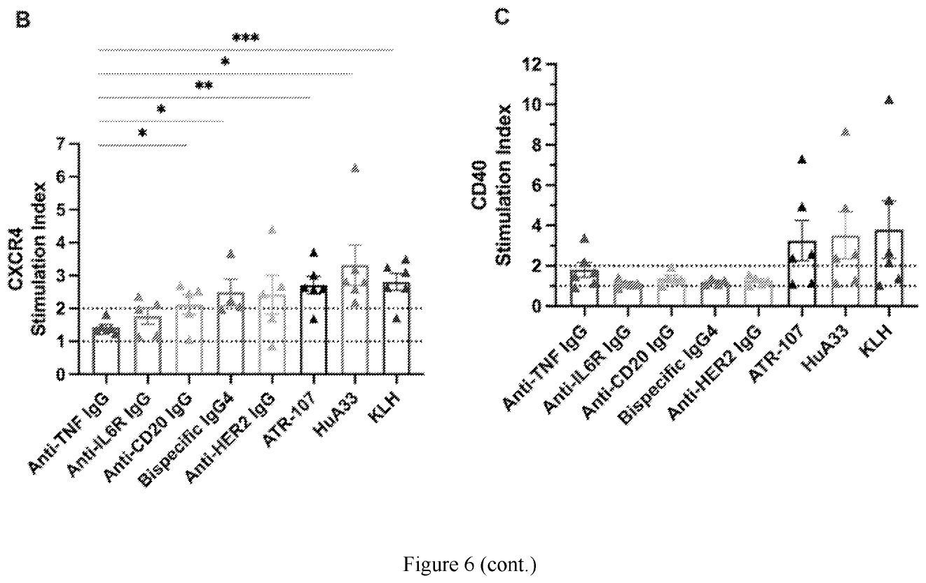

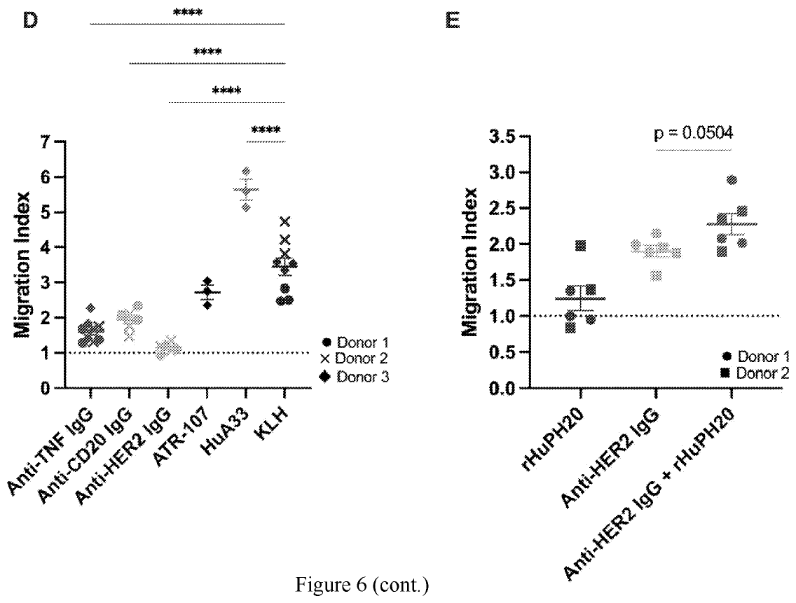

5. moDC were stained for live/dead, HLA-DR, CD11c, CXCR4, and CD40, and then acquired on the BD LSRFortessa. The frequency of CXCR4⁺ moDC in each treatment group was determined in FlowJo (FIG. 6A).

6. In the Transwell migration assay, immature moDC were plated in the upper chambers of 96-well Transwell plates while the lower chambers contained two chemokines in media. Then across the chambers, a low or high concentration gradient of therapeutic protein was established. Cells that migrated into the lower chambers were stained and counted by flow cytometry.

7. Statistical analysis was performed in GraphPad Prism.

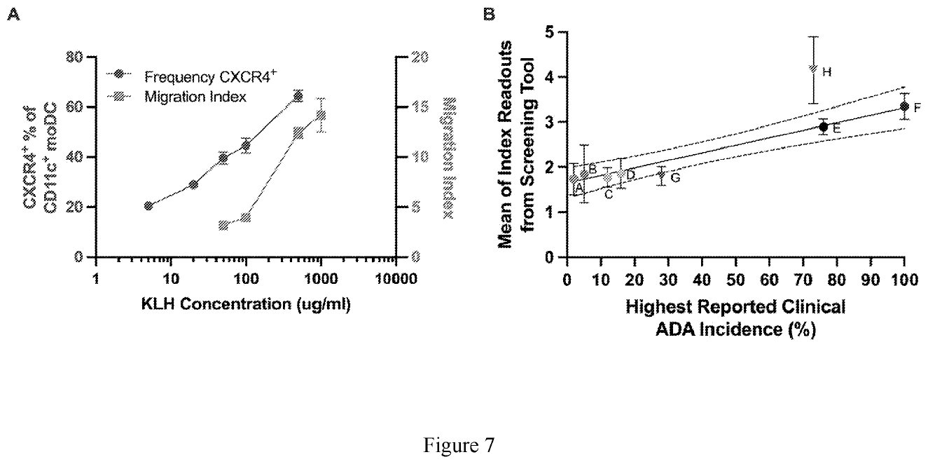

Results. Dendritic cell migratory potential was captured by two in vitro readouts: CXCR4⁺ frequency of moDC and Transwell migration index. First, the change in CXCR4⁺ frequency over the unstimulated treatment group was calculated as ‘Stimulation Index’ for each therapeutic protein. Monoclonal antibodies ATR-107 and HuA33 have high immunogenic potential, and in a population of six healthy donors, ATR-107 and HuA33 strongly upregulated CXCR4 on moDCs with an average stimulation index of 2.71 and 3.31, respectively (FIG. 6B). Also, the co-stimulatory marker CD40 was found to be concurrently upregulated with CXCR4 on moDC (FIG. 6C). Then, in the Transwell migration assay, the migration index was calculated as the fold change in percent migrated moDC toward therapeutic protein and chemokines compared to percent migrated moDC toward chemokines alone. A concentration gradient of ATR-107 and HuA33 induced a migration index of 3-fold and 5.5-fold, respectively (FIG. 6D). However, low to moderate immunogenic potential was predicted by a migration index at or below 2.0. Interestingly, for anti-HER2 IgG, which is co-formulated with hyaluronidase (rHuPH20) in an FDA-approved product, the migration index was slightly increased by the presence of rHuPH20 (FIG. 6E). Readouts of dendritic cell migratory potential were found to be protein-concentration dependent (FIG. 7A). Finally, a strong positive correlation was established between the combined mean of assay readouts and the clinical ADA incidence (FIG. 7B).

The combined readouts of this in vitro screening tool captured dendritic cell migratory potential and correlated with immunogenic risk for multiple therapeutic proteins.

Most notably, HuA33 and ATR-107, which demonstrated high incidence of immunogenicity (73-76%) in early clinical trials, strongly upregulated all markers and induced migration of moDC along a concentration gradient. Results indicate that CXCR4 is indeed a mechanism-based marker to include in preclinical risk assessment.

Furthermore, the Transwell migration assay has a flexible format allowing the testing of dendritic cell migration under many conditions. The migration index readout would be useful in immunogenicity risk assessment of formulation changes and novel modalities, such as gene therapy or nucleic acids.

Although the present disclosure has been described with respect to one or more particular embodiments and/or examples, it will be understood that other embodiments and/or examples of the present disclosure may be made without departing from the scope of the present disclosure.

1. A method for determining dendritic cell migration comprising:
contacting a known amount of dendritic cells with a therapeutic protein at a first protein concentration in a first chamber, wherein the first chamber is connected to and adjacent to a second chamber and the first chamber and second chamber are separated by a porous membrane, wherein the second chamber contains the therapeutic protein at a second protein concentration and one or more chemokines and does not contain any dendritic cells;
incubating the first chamber and second chamber;
isolating migrated dendritic cells from the second chamber;
staining the migrated dendritic cells from the second chamber; and
counting the migrated dendritic cells from the second chamber,
wherein the counted cells have migrated from the first chamber.

2. The method according to claim 1, wherein the first protein concentration is lower than the second protein concentration.

3. The method according to claim 2, wherein the first protein concentration is fivefold or tenfold less than the second protein concentration.

4. The method according to claim 1, wherein the first protein concentration is 0 to 250 μg/mL.

5. The method according to claim 4, wherein the first protein concentration is about or is 100 μg/mL.

6. The method according to claim 1, wherein the second protein concentration is 50 to 50,000 μg/mL.

7. The method according to claim 6, wherein the second protein concentration is about or is 1000 μg/mL.

8. The method according to claim 1, wherein the second chamber contains at least two different chemokines.

9. The method according to claim 8, wherein there are two chemokines and the chemokines are CCL21 and CXCL12.

10. The method according to claim 8, wherein there are three chemokines and the chemokines are CCL19, CCL21, and CXCL12.

11. The method according to claim 8, wherein the concentration of each chemokine is 1 to 200 ng/mL.

12. The method according to claim 11, wherein the concentration of each chemokine is about or is 100 ng/mL.

13. The method according to claim 1, wherein prior to migration the first chamber comprises about 10,000 to about 30,000 dendritic cells.

14. The method according to claim 13, wherein prior to migration the first chamber comprises about 12,000±1000 dendritic cells.

15. The method according to claim 1, wherein the therapeutic protein is suspected of inducing an immunogenic response in an individual.

16. The method according to claim 1, wherein the therapeutic protein is a monoclonal antibody.

17. The method according to claim 1, wherein the therapeutic protein is recombinant enzymes, cytokines, globular proteins, AAV capsid proteins, antibody-drug conjugates, or any combination thereof.

18. The method according claim 1, wherein the counting is performed by flow cytometry.

19. The method according to claim 1, wherein the porous membrane has a pore size of 5 to 10 μm.

20. The method according to claim 1, further comprising determining the percent of dendritic cells that migrated.

21. The method according to claim 1, further comprising determining a migration index by comparing the percent of migrated cells to a percent of migrated cells from a control group.

22. The method according to claim 1, wherein the method utilizes a transwell plate comprising one or more Boyden chambers.
